(19) United States
(12) Patent Application Publication
(10) Pub. No.: US 2025/0213121 A1
(43) Pub. Date: Jul. 3, 2025

(54) System for Facilitating a Remote Medical Examination

(71) Applicant: Bethune Alafia Grindley, Stephens City, VA (US)

(72) Inventor: Bethune Alafia Grindley, Stephens City, VA (US)

(21) Appl. No.: 19/003,773

(22) Filed: Dec. 27, 2024

Related U.S. Application Data
(60) Provisional application No. 63/615,157, filed on Dec. 27, 2023.

(51) Int. Cl. A61B 5/0205; A61B 3/12; A61B 5/00; A61B 5/022; A61B 5/107; A61B 7/02; G01G 19/44; G01G 19/50

(52) U.S. Cl. CPC A61B 5/0205; A61B 5/6831; A61B 5/6843; A61B 5/7465; G01G 19/445; G01G 19/50; A61B 3/12; A61B 5/0077; A61B 5/02233; A61B 5/1072; A61B 7/02; A61B 2560/0443; A61B 2562/066; A61B 2562/164

(57) ABSTRACT

A system for facilitating a remote medical examination is a system for the remote collection and storage of a patient's physical examination data for augmenting clinical evaluation and clinical decision-making of medical staff. The system includes a flexible housing, examination modules, a controller, a portable power source, a wireless communication module, and an examination monitoring device. The flexible housing corresponds to main structure of the system that supports the operation of the other components. The examination modules includes several medical examination instruments that allow the monitoring and recording of different patient's health vitals. The controller and the portable power source enable the autonomous or semiautonomous operation of the system. The wireless communication module enables the wireless transmission of the patient's health vital data captured by the examination modules to the examination monitoring device. The examination monitoring device enables the patient data to be uploaded to the corresponding healthcare system.

### FIELD OF THE INVENTION

The present invention relates generally to medical devices and telemedicine services. More specifically, the present invention discloses a system that facilitates remote physical examination and monitoring of a patient.

### BACKGROUND OF THE INVENTION

A medical device that allows remote examination of a patient is in demand. Modern advancements in healthcare have brought about dramatic improvements to medical devices. Modern medical devices have many different applications, with single health monitoring device being able to determine a wide variety of information about a patient. However, such monitoring devices often feature complex user configurations that can be complicated for the user or medical staff to use. For example, monitoring devices may utilize communications links to various peripheral components, such as sensors and other medical hardware, that provide the interfaces and functionality needed to monitor a person's health. More complicated monitoring devices are rarely, if ever, used for home care, patients who need to use such devices are usually transported to a doctor's office or other diagnostic facility for evaluation and treatment. The transportation of said device and the patient follow-up through home visits by doctors or nurses, is sporadic, time-consuming, and generally very expensive.

As a result, continuous detailed monitoring of patients is very rare, except in fortuitous circumstances in times of emergency. For example, when a patient has an infectious disease or is infected with a virus, medical staff examining the patient run the risk of contracting or transmitting the infection or virus when the medical staff is physically present with the patient. Because virus monitoring, detection, and identification poses many health risks to healthcare personnel and even the public, systems for remotely examining and monitoring a patient's condition are needed, but currently available devices for monitoring patients' heart rate and respiratory rate are limited in their capacity and do not provide the other needed physical examination data required for thorough evaluation of the respiratory, cardiovascular, or abdominal examinations. A device with capacity for remote examination in the home and routine hospital setting would be useful.

Therefore, an objective of the present invention is to provide a system for facilitating a remote medical examination that addresses the limitations of current medical monitoring devices. The present invention implements an innovative remote monitoring system that is designed to provide portability and convenience of installation so that a medical examination can be performed remotely. The present invention enables a touch-less examination that reduces the transmission of Multi-Drug-Resistant Organisms (MDROs) and other infections that can be transmitted between patients/people, protects healthcare workers from morbidity and mortality associated with contracting these infections, and reduces the need for multiple examinations of a single patient by multiple managing healthcare providers as objective examination data is collected and stored. Further, the present invention impacts the physician shortage by augmenting the extension of physician services while helping to ensure quality thereof and by helping recruit immunocompromised medical providers back to a workforce experiencing critical shortage of healthcare providers as those providers would be shielded from infection risks. Another objective of the present invention is to provide a system that includes different monitoring and medical examination tools that allow the remote medical examination of the patient. The present invention can include a wearable examination device that can be easily put on by the patient to perform the remote medical examination. The present invention can also be implemented on different furniture, such as a bed frame, to accommodate the patient's medical needs. Further, the present invention can include spare auscultation units which are available to be applied independently as adhering discs. Additional features and benefits of the present invention are further discussed in the sections below.

### SUMMARY OF THE INVENTION

The present invention discloses a system for facilitating a remote medical examination. The system of the present invention enables the remote collection and storage of physical examination data for augmenting clinical evaluation and clinical decision-making of medical staff. The system of the present invention includes different patient examination devices configured to generate the physical examination data based on measuring at least one physiological parameter of the patient. In the preferred embodiment, the system of the present invention includes at least one patient examination device designed to be worn around the patient's torso to physically examine different health vitals using different medical examination technologies. The patient examination device can include several examination modules arranged to measure different health vitals when worn by the patient including, but not limited to, heart and respiratory rates, heart sounds, breath sounds, adventitious breath sounds (such as crackles, wheezes rhonchi), bowel sounds, oxygen saturation, etc.

The patient examination device of the present invention can be provided in different configurations to accommodate different medical examination requirements. For example, the patient examination device of the present invention can be provided as a wearable device that can be worn around the patient's torso. Different fastening mechanisms can be implemented to help secure the patient examination device to the user's body so that the remote medical examination can be performed automatically. In another embodiment, the patient examination device can be implemented on different external structures, such as a bed frame. This way, the patient can be remotely examined while resting on a bed, which is beneficial in situations where the patient is bed bound. In this embodiment, the patient examination device can be molded into a C-shaped or U-shaped structure that can be placed around the abdomen during the examination process to evaluate bowel sounds and distention via novel pressure evaluation device.

Further, the system of the present invention can include additional medical examination instruments that can be utilized along with the patient examination device to perform the medical examination process. For example, the system of the present invention may include a volume status evaluator for edema, an examination camera in the vicinity of the examination, etc. The examination camera can be implemented to perform various functions including, but not limited to, detecting pallor, cyanosis, icterus, fever and other abnormalities, tremors, obvious masses, wounds, respiratory rate, obvious bleeding, speech quality, and limb movements.

### BRIEF DESCRIPTION OF THE DRAWINGS

FIG. 1 is a top-front-left perspective view of the patient examination device of the system of the present invention, wherein the patient examination device is shown in a flat configuration.

FIG. 2 is a bottom-rear-right perspective view of the patient examination device of the system of the present invention.

FIG. 3 is a front view of the patient examination device of the system of the present invention.

FIG. 4 is a top-front-right perspective view of the patient examination device of the system of the present invention, wherein the patient examination device is shown in a worn configuration.

FIG. 5 is a top view of the patient examination device of the system of the present invention.

FIG. 6 is a top-front-left perspective view of an alternate embodiment of the system of the present invention, wherein the patient examination device is shown implemented on a bed frame.

FIG. 7 is a front view of the alternate embodiment of the system of the present invention.

FIG. 8 is a side schematic view of the alternate embodiment of the system of the present invention.

FIG. 9 is a front view of the alternate embodiment of the patient examination device of the system of the present invention.

FIG. 10 is a box diagram of the system of the present invention showing the electrical connections and the electrical connections, wherein the electrical connections are shown in solid lines, and wherein the electronic connections are shown in dashed lines.

FIG. 11 is a side schematic view of an alternate embodiment of the examination present invention, wherein the patient examination device is shown with a portable air pump, wherein the portable air pump is shown in fluid communication with the examination modules, and wherein the fluid connections are shown in dot-dash lines.

FIG. 12 is a box diagram of the system of the present invention showing additional medical examination instruments of the system, wherein the medical examination instruments are shown communicably coupled to the examination monitoring device.

FIG. 13 is a front schematic view of the patient examination device of the system of the present invention, wherein the patient examination device is shown worn by a patient.

FIG. 14 is a schematic view of the overall system of the present invention.

FIG. 15 is a box diagram showing the examination monitoring device of the system of the present invention.

FIG. 16 is a box diagram showing the general device configuration of the examination monitoring device and the provider examination device of the system of the present invention.

### DETAIL DESCRIPTIONS OF THE INVENTION

All illustrations of the drawings are for the purpose of describing selected versions of the present invention and are not intended to limit the scope of the present invention.

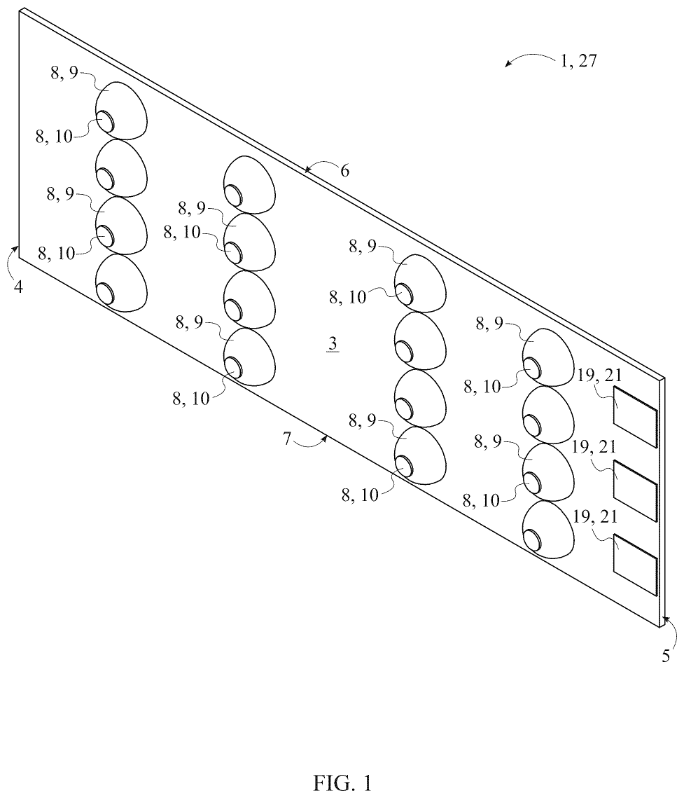

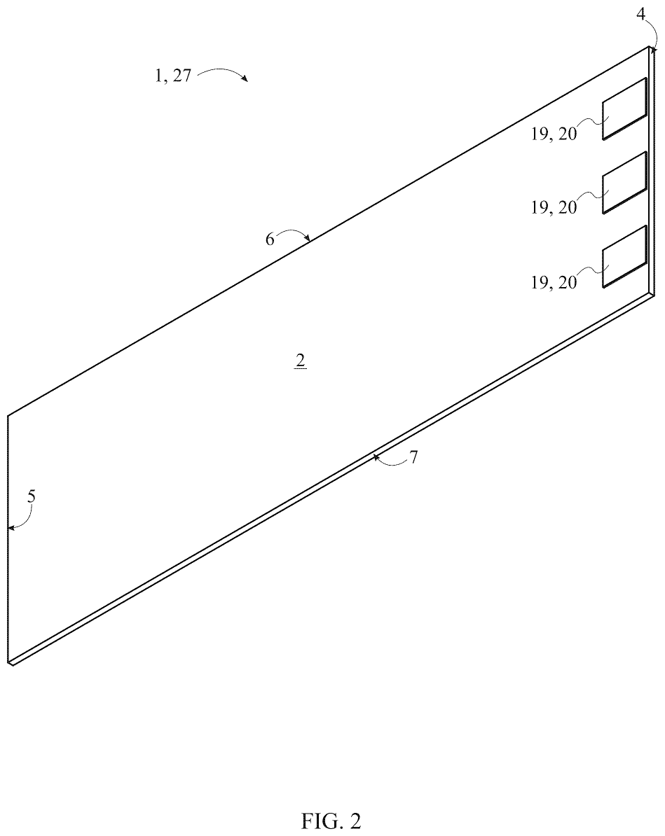

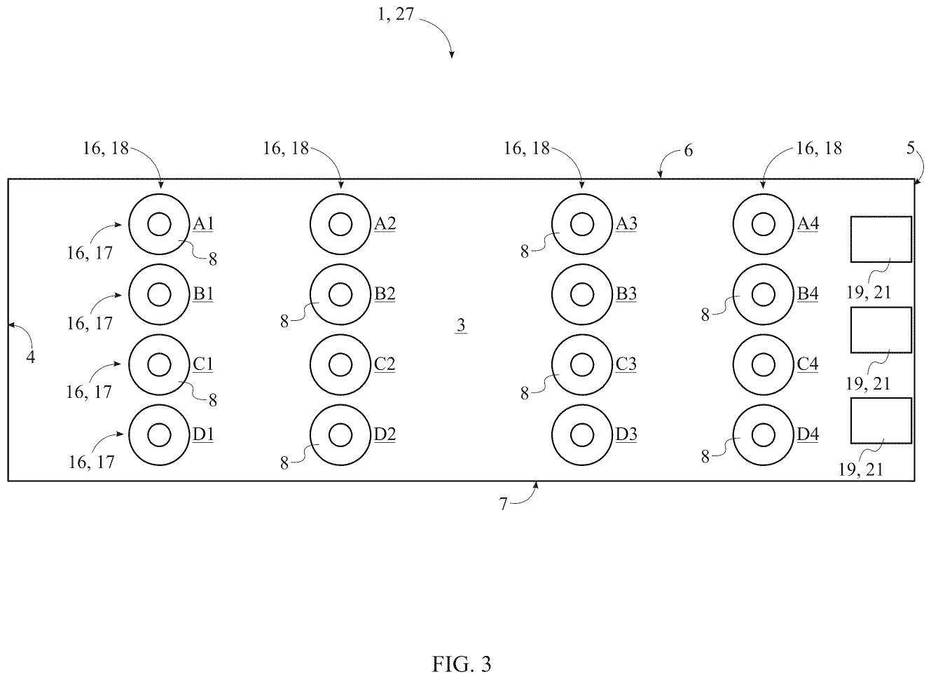

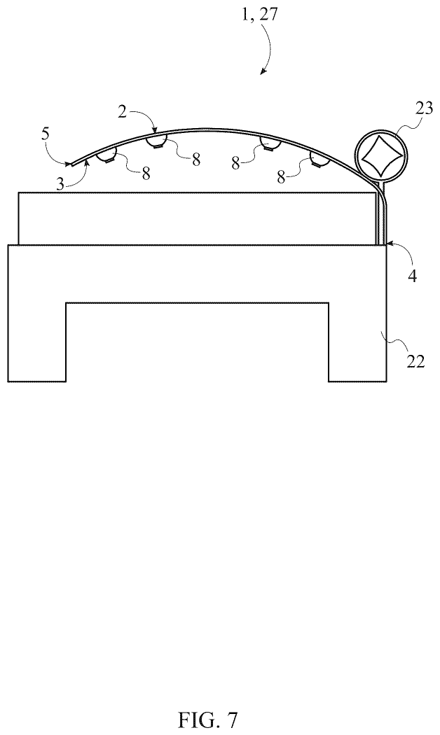

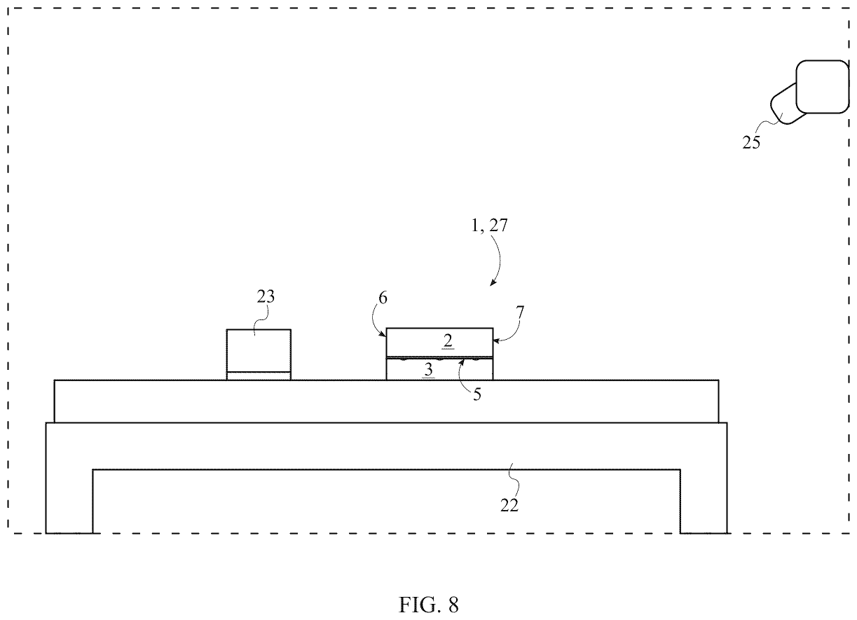

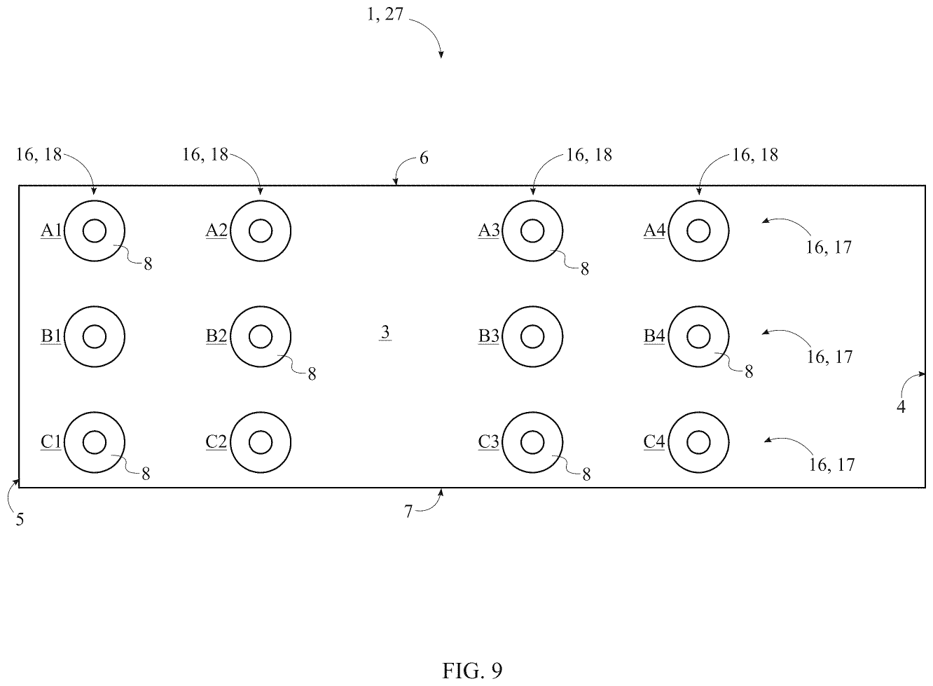

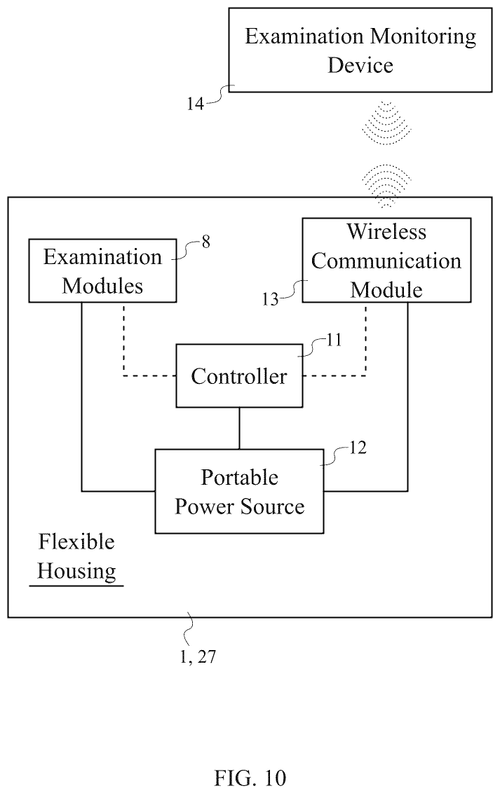

The present invention discloses a system for facilitating a remote medical examination. As can be seen in FIG. 1 through 13, the system of the present invention enables at least one patient examination device to remotely collect and store physical examination data for augmenting clinical evaluation and clinical decision-making of medical staff. The patient examination device 27 allows remote medical examinations to minimize the risk of disease transmission while enhancing the effectiveness of telemedicine. The patient examination device 27 also increases the efficiency of in-person medicine especially in organizations caring for patients with Multi-Drug-Resistant Organisms (MDROs). Because healthcare personnel need not be present to perform the examination process, the patient examination device 27 minimizes the risk of transmitting severe and life-threatening communicable diseases such as Ebola, COVID-19, influenza, etc. Healthcare personnel can be in close proximity, for example, outside the room but not in contact with the patient.

As can be seen in FIG. 1 through 5, 10, and 13, in the preferred embodiment, the system of the present invention comprises a flexible housing 1, a plurality of examination modules 8, a controller 11, a portable power source 12, a wireless communication module 13, and an examination monitoring device 14. The flexible housing 1 corresponds to main structure of the patient examination device 27 that supports the operation of the other components. The plurality of examination modules 8 includes several medical examination instruments that allow the monitoring and recording of different patient's health vitals. The controller 11 and the portable power source 12 enable the autonomous or semiautonomous operation of the patient examination device 27. The wireless communication module 13 enables the wireless transmission of the patient's health vital data captured by the plurality of examination modules 8 to the examination monitoring device 14. The examination monitoring device 14 enables the patient data to be uploaded to the corresponding healthcare system. Patient data includes, but is not limited to, heart sounds, breath sounds, bowel sounds, and other examination data.

The general configuration of the aforementioned components enables the remote medical examination of a patient in an efficient and practical manner. As can be seen in FIG. 1 through 5, 10, and 13, the flexible housing 1 is designed to enable to be positioned around the patient's target body area to monitor and record the necessary health vitals data. In general, the flexible housing 1 is designed as an elongated rectangular structure with a size large enough to cover the target body area. So, the flexible housing 1 comprises an outer housing surface 2, an inner housing surface 3, a first lateral edge 4, a second lateral edge 5, a proximal housing edge 6, and a distal housing edge 7. The inner housing surface 3 preferably corresponds to the surface that is exposed to the patient's body, while the outer housing surface 2 corresponds to the surface that is oriented away from the patient's body. The first lateral edge 4 and the second lateral edge 5 correspond to the opposite lateral edges that match the widthwise edges of the flexible housing 1. Similarly, the proximal housing edge 6 and the distal housing edge 7 correspond to the opposite lateral edges the match the lengthwise edges of the flexible housing 1. The flexible housing 1 and the examination monitoring device 14 are preferably designed to be reusable and recyclable after disinfection with any suitable cleaning solutions, such as ethylene oxide. In some embodiments, the examination monitoring device 14 may include a serial number and corresponding Identification number (ID) recorded in the data storage component of the examination monitoring device 14. The serial number and ID can allow healthcare personnel to deliver the examination monitoring device 14 to patients with instructions for use while allowing remote identification of the delivered examination monitoring device 14. Further, the serial number and the ID of the patient examination device 27 enables accurate pairing to patient records as a unique identifier designated to each patient examination device 27 of the corresponding patient.

In the preferred embodiment, the patient examination device 27 of the present invention can be implemented as follows: the proximal housing edge 6 and the distal housing edge 7 are positioned opposite to each other across the flexible housing 1 due to the overall width of the flexible housing 1, as can be seen in FIG. 1 through 5, 10, and 13. Likewise, the first lateral edge 4 and the second lateral edge 5 are positioned opposite to each other across the flexible housing 1 due to the overall length of the flexible housing 1. Further, the first lateral edge 4 and the second lateral edge 5 are positioned in between the proximal housing edge 6 and the distal housing edge 7 as the flexible housing 1 is preferably designed as a single solid body. In addition, the plurality of examination modules 8 is distributed throughout and across the inner housing surface 3. The distribution and arrangement of the plurality of examination modules 8 is determined according to the application of the patient examination device 27 to target specific body areas. Further, each of the plurality of examination modules 8 is externally integrated onto the inner housing surface 3 to secure each of the examination modules to the flexible housing 1. This way, the plurality of examination modules 8 can be manipulated and positioned by handling the flexible housing 1. Further, the controller 11, the portable power source 12, and the wireless communication module 13 are mounted within the flexible housing 1 to protect the corresponding electronic and the electrical components from the surroundings. Alternatively, if needed for unique cases requiring individualized modification of the physical examination, individual units of the examination modules 8 can be provided for direct attachment to the patient's body. For example, individual auscultation units can be applied independently as adhering discs to the additional locations required by the examining provider.

As can be seen in FIG. 1 through 5, 10, and 13 through 16, on the other hand, the examination monitoring device 14 is positioned offset to the flexible housing 1. The examination monitoring device 14 can be a portable computing device that allows the patient or healthcare provider to monitor and control the operation of the patient examination device 27. The examination monitoring device 14 can be provided separate from the flexible housing 1 or as a detachable accessory. Further, the plurality of examination modules 8 and the wireless communication module 13 are electronically connected to the controller 11. This way, the captured health vital data from the plurality of examination modules 8 can be transmitted to the controller 11 for processing, which can then be relayed to the wireless communication module 13 for transmission of the patient's health data to the examination monitoring device 14. Further, the plurality of examination modules 8, the wireless communication module 13, and the controller 11 are electrically connected to the portable power source 12. This way, the electrical power necessary for the operation of the corresponding electrical and electronic components can be transmitted. Finally, the wireless communication module 13 is communicably coupled with the examination monitoring device 14 to enable the wireless transmission of the patient's health data from the patient examination device 27 to the examination monitoring device 14. This way, the patient's health data can be relayed to the corresponding healthcare system that the appropriate healthcare staff can access during the examination process.

Each of the plurality of examination modules 8 is designed to enable the proper examination of the patient's target health vitals during the examination process. As can be seen in FIG. 1 through 5, 10, and 13, each of the plurality of examination modules 8 comprises a support base 9 and a health vital monitoring device 10. The support base 9 is preferably designed as a dome-shaped support base 9 that positions the health vital monitoring device 10 against the patient's body. Each health vital monitoring device 10 is a medical instrument designed to monitor one or more health vitals. Each health vital monitoring device 10 is preferably a micro stethoscope, but other medical instruments can be implemented. In general, each examination module can be implemented as follows: the support base 9 is mounted onto the inner housing surface 3 to secure the support base 9 to the flexible housing 1. The flat face of the dome-shaped support base 9 is preferably connected to the flexible housing 1 using the appropriate manufacturing methods, while the convex face of the dome-shaped support base 9 is oriented away from the flexible housing 1. Further, the health vital monitoring device 10 is centered on the support base 9. The health vital monitoring device 10 is preferably centered on the convex face of the dome-shaped support base 9 to position the health vital monitoring device 10 offset from the flexible housing 1. Furthermore, the health vital monitoring device 10 is mounted onto the support base 9, opposite the inner housing surface 3 to secure the health vital monitoring device 10 to the support base 9.

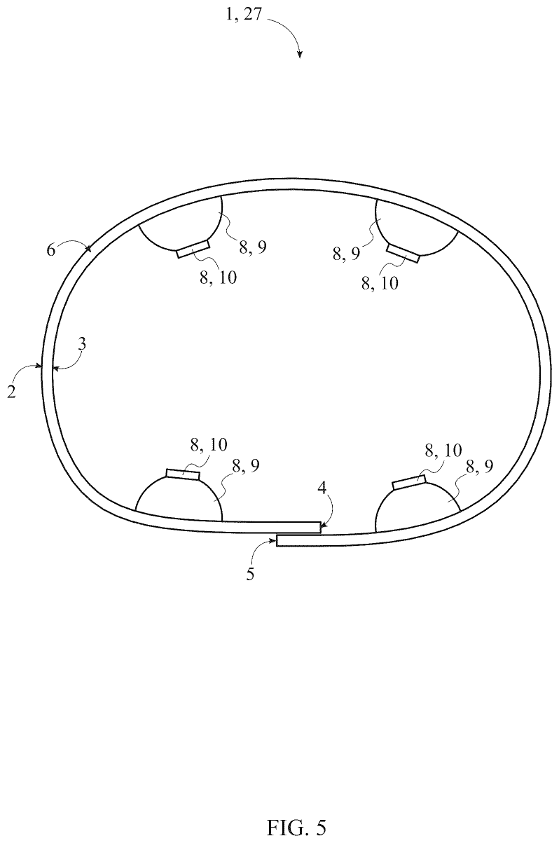

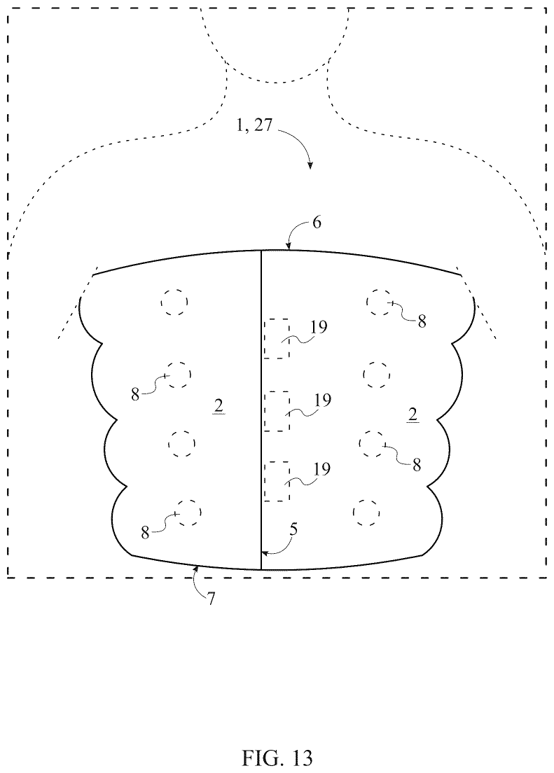

In the preferred embodiment, the support base 9 is a soft structure that is comfortable and safe to the patient's body. As can be seen in FIGS. 5 and 13, when the flexible housing 1 is placed on the patient's body, with the inner housing surface 3 against the patient's body, the soft structure of the support base 9 pressures the corresponding health vital monitoring device 10 against the user's body to facilitate the proper monitoring of the target health vitals. The support base 9 is preferably a pneumatic base that has been prefilled with a predetermined amount of consumer-face gas, such as air, so that the support base 9 retains the dome shape. However, in some embodiments, the patient examination device 27 can include means to facilitate the inflation of the support base 9 of each of the plurality of examination modules 8.

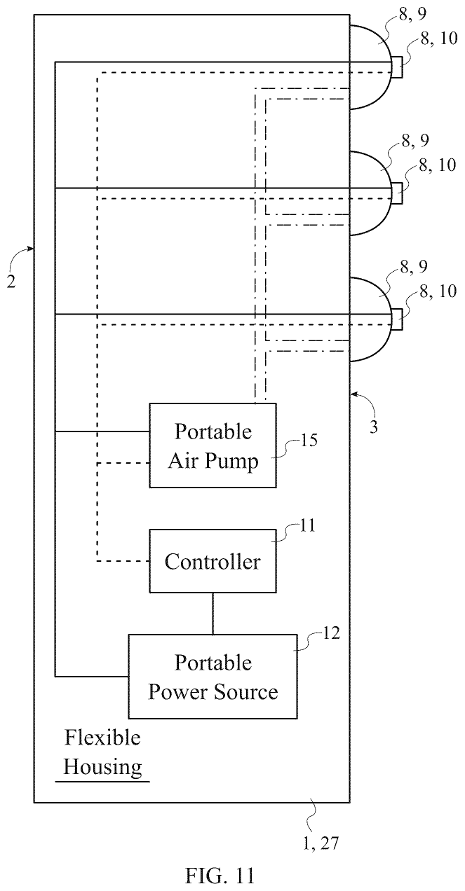

As can be seen in FIGS. 11 and 13, to enable the selective inflation of each support base 9, the system of the present invention may further comprise a portable air pump 15 that allows the patient or the healthcare provider to inflate the support base 9 of each of the plurality of examination modules 8 prior to the patient's examination. The portable air pump 15 is preferably integrated into the flexible housing 1 so that the portable air pump 15 is carried along with the flexible housing 1. Further, the portable air pump 15 is in fluid communication with the support base 9 of each of the plurality of examination modules 8 so that the air pumped by the portable air pump 15 flows into each support base 9. If a different gas is utilized, a container holding a predetermined amount of gas can be implemented which can be refilled as necessary. Further, the portable air pump 15 is electronically connected to the controller 11 to allow the user to control the operation of the portable air pump 15 from the examination monitoring device 14. In addition, the portable air pump 15 is electrically connected to the portable power source 12 to transmit the electrical power necessary for the operation of the portable air pump 15. In other embodiments, different mechanisms can be implemented to enable the controlled inflation of the support base 9 of each of the plurality of examination modules 8 prior to the examination process.

As previously discussed, the patient examination device 27 can be provided in different embodiments to enable the system to be utilized in different manners. As can be seen in FIG. 1 through 5 and 13, in one embodiment, the patient examination device 27 can be provided as a wearable device that can be worn around the patient's torso. The wearable embodiment of the patient examination device 27 serves as a remote lung and heart auscultation device. In the wearable embodiment of the patient examination device 27, the plurality of examination modules 8 is arranged to be positioned around the chest area of the patient to target the posterior and anterior regions of the chest area. To do so, the plurality of examination modules 8 can be arranged into a rectangular array 16 that evenly distributes the plurality of examination modules 8 on the inner housing surface 3. The rectangular array 16 comprises a plurality of modules rows 17 and a plurality of modules columns 18.

As can be seen in FIG. 1 through 5 and 13, in the wearable embodiment of the patient examination device 27, the plurality of modules rows 17 includes four rows of modules, and the plurality of modules columns 18 includes four columns of modules. This results in a rectangular array 16 of sixteen examination modules. Further, the plurality of modules rows 17 is distributed from the proximal housing edge 6 to the distal housing edge 7 to evenly distribute the plurality of modules rows 17 along the width of the flexible housing 1. In addition, the plurality of modules columns 18 is distributed from the first lateral edge 4 to the second lateral edge 5 to evenly distribute the plurality of modules columns 18 along the length of the flexible housing 1. This rectangular array 16 can be labeled in a specific way for control purposes: the four modules rows can be designated as row A, row B, row C, and row D. Row A preferably corresponds to the modules row adjacent to the proximal housing edge 6, row D corresponding to the modules row adjacent to the distal housing edge 7, and row B and row C corresponding to the intermediate modules rows. Similarly, the four modules columns can be designated as column 1, column 2, column 3, and column 3. Column 1 corresponds to the modules column adjacent to the first lateral edge 4, column 4 corresponds to the modules column adjacent to the second lateral edge 5, and column 2 and column 3 correspond to the intermediate modules columns. The rectangular array 16 allows specific health vitals to be monitored. For example, the examination module A4 and B4 can enable the auscultation of hear sounds in the mitral area. Further, column 1 allows auscultation in the right anterior chest, column 2 allows auscultation in the right posterior chest, column 3 allows auscultation in the left posterior chest, and column 4 allows auscultation in the left anterior chest. In other embodiments, the rectangular array 16 of the plurality of examination modules 8 can include a different number of modules rows and modules columns to target additional body areas.

Further, to enable the patient examination device 27 to be comfortably and securely worn by the patient, the system of the present invention may further comprise a housing fastener 19, as can be seen in FIG. 1 through 5 and 13. The housing fastener 19 is preferably an interlocking fastener that allows the patient or the healthcare provider to secure the patient examination device 27 to the user's body. The housing fastener 19 is preferably a hook-and-loop fastener, but other fasteners can be implemented. In addition, the housing fastener 19 comprises a plurality of first fastener pieces 20 and a plurality of second fastener pieces 21 corresponding to several interlocking pieces of the housing fastener 19. The plurality of first fastener pieces 20 corresponds to the loop portion of the hook-and-loop fastener while the plurality of second fastener pieces 21 corresponds to the hook portion. To implement the housing fastener 19, the plurality of first fastener pieces 20 is distributed along the first lateral and each of the plurality of first fastener pieces 20 is mounted onto the outer housing surface 2, adjacent to the first lateral edge 4. This way, each of the plurality of first fastener pieces 20 is secured to the outer housing surface 2 along the first lateral edge 4. On the other hand, the plurality of second fastener pieces 21 is distributed along the second lateral edge 5 and each of the plurality of second fastener pieces 21 is mounted onto the inner housing surface 3, adjacent to the second lateral edge 5. This way, each of the plurality of second fastener pieces 21 is secured to the inner housing surface 3 along the second lateral edge 5. Thus, to secure the flexible housing 1 to the patient's body, each second fastener piece is engaged to the corresponding first fastener piece. In other embodiments, the housing fastener 19 can be implemented differently according to the type of fastener being utilized.

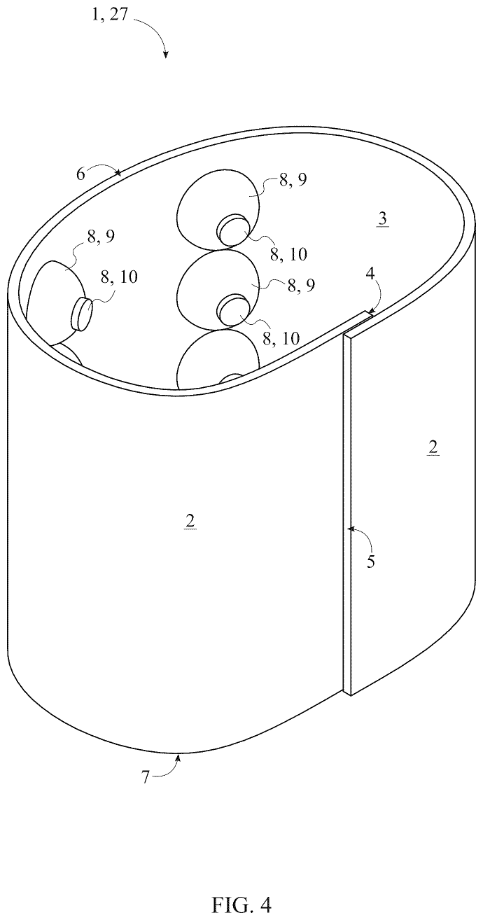

In the wearable embodiment, the patient examination device 27 may be used as follows: the patient places and fastens the flexible housing 1 around the thorax using the housing fastener 19, as can be seen in FIGS. 4, 5, and 13. The examination monitoring device 14 is then activated to engage the plurality of examination modules 8. Then, the patient breathes normally as the patient would for an in-person examination. The sounds recorded by the plurality of examination modules 8 during a predetermined period of time (e.g. 30 seconds) are then transmitted to the healthcare personnel via the examination monitoring device 14. Different communication protocols can be implemented to enable the secure transmission of the patient's health data. For example, the communication protocols can include, but are not limited to, wired or wireless networks, third-party communication services, etc. Examples of wireless communications include, but are not limited to, Radio Frequency (RF) technologies, infrared, Bluetooth, Wireless Local Area Network (WLAN), wireless radio networks such as a Long-Term Evolution (LTE) networks, WiMAX network, 3G wireless networks, 4G wireless networks, 5G wireless networks, etc.

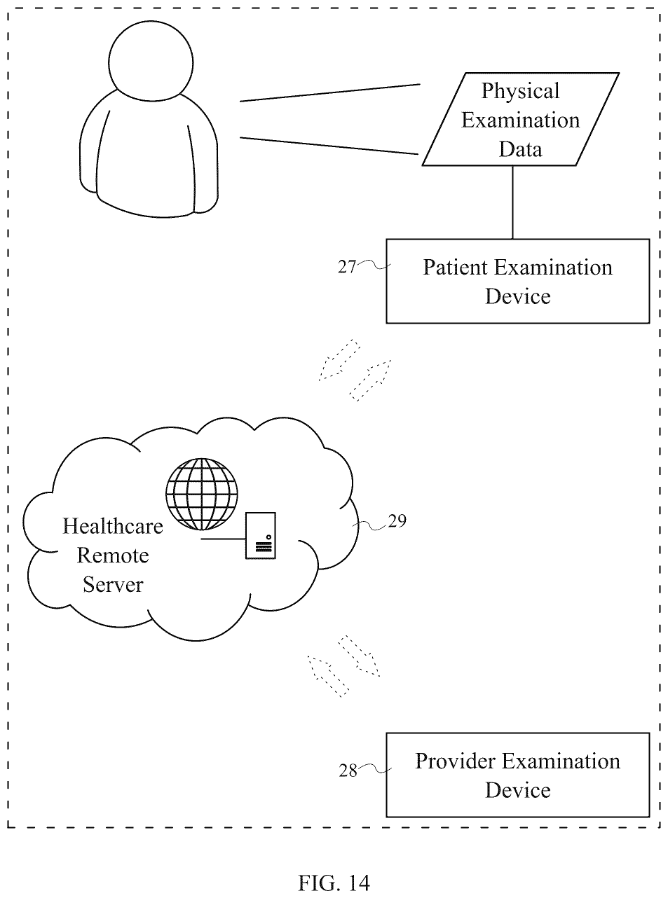

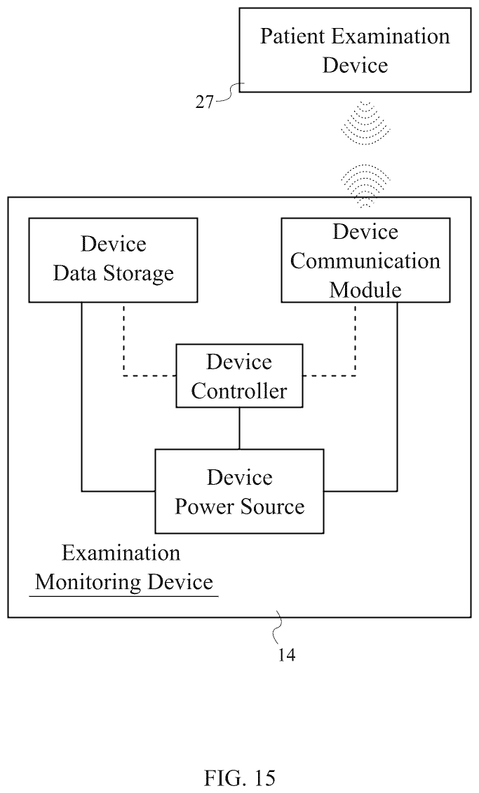

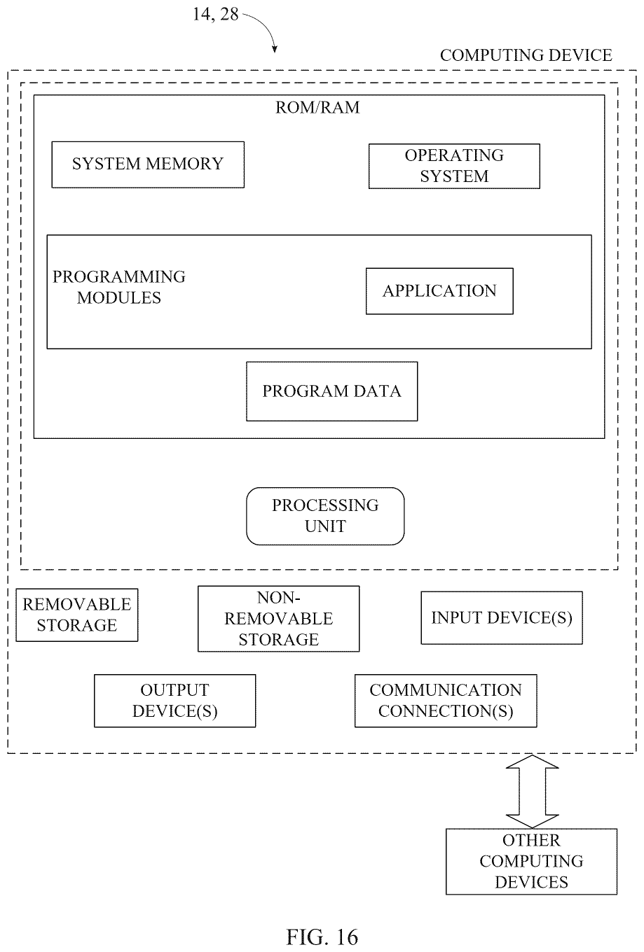

The use of wireless communication protocols enable the healthcare provider to listen to the captured data during the examination, such lung and heart sounds, in real time. Further, all captured data is stored at the healthcare database to facilitate continuous monitoring of the progression of illnesses during clinical management. For example, the examination of stored examination data can help detect a changing murmur in infective endocarditis, evolving lung sounds as a pneumonia progresses, improvement in wheezing and zones affected in managing an exacerbation of asthma, etc. As can be seen in FIG. 14 through 16, the system of the present invention may further comprise a provider examination device 28 and a healthcare remote server 29. The provider examination device 28 is a computing device that allows the healthcare provider to monitor the examination process. For example, the provider examination device 28 can be a wearable wireless earpiece that allows the healthcare provider to listen to the transmitted data signals (e.g., lung, heart, bowel sounds, etc.). In addition, the at least one healthcare remote server 29 includes at least one healthcare database that stores all the collected data from the patient examination device 27. The provider examination device 28 and the examination monitoring device 27 are communicably coupled with the at least one healthcare remote server 29 so that the captured data from the patient examination device 27 is relayed to the at least one healthcare remote server 29. When the captured data is received, the captured data is processed and stored at the at least one healthcare database for future accessibility. Further, the captured data is relayed from the healthcare remote server 29 to the provider examination device 28 so that the healthcare provider can monitor the remote examination process. In other embodiments, different computing systems can be implemented to enable remote monitoring of the examination process by the healthcare provider in real time.

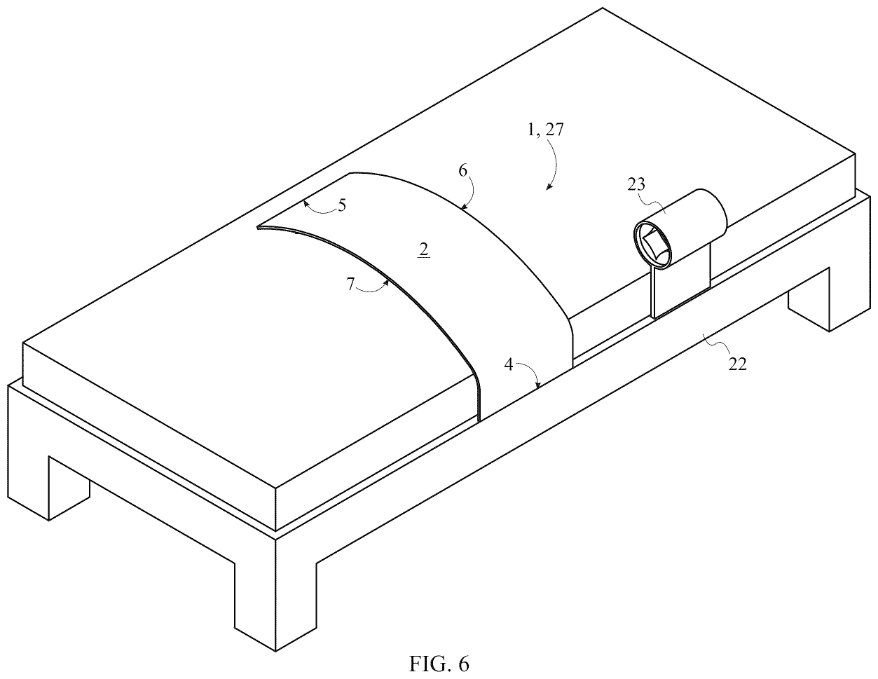

In another embodiment, the system of the present invention can be implemented on an external structure that supports the patient's body. For example, the system of the present invention can be implemented on a piece of furniture, such as a medical bed, to enable the remote examination of a bed-bound patient. As can be seen in FIG. 6 through 9, in this embodiment, the system of the present invention may further comprise a bed frame 22 that accommodates the mattress to support the patient's body. In the bed embodiment of the present invention, the patient examination device 27 is modified to position the plurality of examination modules 8 in such a way that each examination module can reach the patient's target body areas while the patient is laying on the bed. To do so, the flexible housing 1 is shaped into a C-shaped flexible housing 1 so that the flexible housing 1 can protrude past the bed mattress from a side of the bed frame 22 and reach the patient's target body areas.

Like the wearable embodiment, the bed embodiment of the patient examination device 27 is designed to cover a wide area of the patient's body during the examination process. As can be seen in FIG. 6 through 9, the bed embodiment of the patient examination device 27 is preferably designed to cover the abdominal area of the patient's body while the patient is laying on the bed. However, the patient examination device 27 can be altered to cover other body areas when the patient is laying on the bed. The bed embodiment of the patient examination device 27 evaluates distention and tenderness, evaluates bowel sounds, and evaluates distention. To cover the abdominal area of the bed-bound patient, the rectangular array 16 of the plurality of examination modules 8 is arranged differently to accommodate the bed frame 22. In the bed embodiment of the patient examination device 27, the plurality of modules rows 17 is distributed from the proximal housing edge 6 to the distal housing edge 7 to evenly distribute the plurality of module rows along the width of the flexible housing 1.

As can be seen in FIG. 6 through 9, the plurality of modules columns 18 is also distributed from the first lateral edge 4 to the second lateral edge 5 to distribute the plurality of modules columns 18 along the length of the flexible housing 1. However, the rectangular array 16 is positioned offset to the first lateral edge 4 to leave an empty space on the flexible housing 1 that accommodates the bed frame 22. Furthermore, the first lateral edge 4 is laterally connected to the bed frame 22 to secure the flexible housing 1 to the bed frame 22. Unlike the wearable embodiment of the patient examination device 27, in the bed embodiment the plurality of modules rows 17 includes three rows of modules, and the plurality of modules columns 18 includes four columns of modules. The rectangular array 16 of the bed embodiment enables the evaluation of all quadrants of the lateral and anterior abdomen areas for tenderness and audio evaluation of bowel sounds. In other embodiments, the rectangular array 16 of the plurality of examination modules 8 may include a different number of modules rows and/or modules columns.

As can be seen in FIG. 6 through 9 and 12, the bed embodiment of the system of the present invention may include additional medical examination instruments implemented on the bed frame 22. In some embodiments, the system of the present invention may further comprise an electronic sphygmomanometer 23. The electronic sphygmomanometer 23 is a slidable sphygmomanometer that the patient can use while lying on the bed. The electronic sphygmomanometer 23 is positioned offset to the flexible housing 1 to not obstruct the operation of the patient examination device 27. Further, the electronic sphygmomanometer 23 is laterally mounted onto the bed frame 22 to secure the electronic sphygmomanometer 23 to the bed frame 22. The electronic sphygmomanometer 23 can be mounted on the bed frame 22 in a fixed manner or in a detachable manner as necessary. Furthermore, the electronic sphygmomanometer 23 is communicably coupled with the examination monitoring device 14 so that the patient's health vitals monitored by the electronic sphygmomanometer 23 can also be relayed to the examination monitoring device 14.

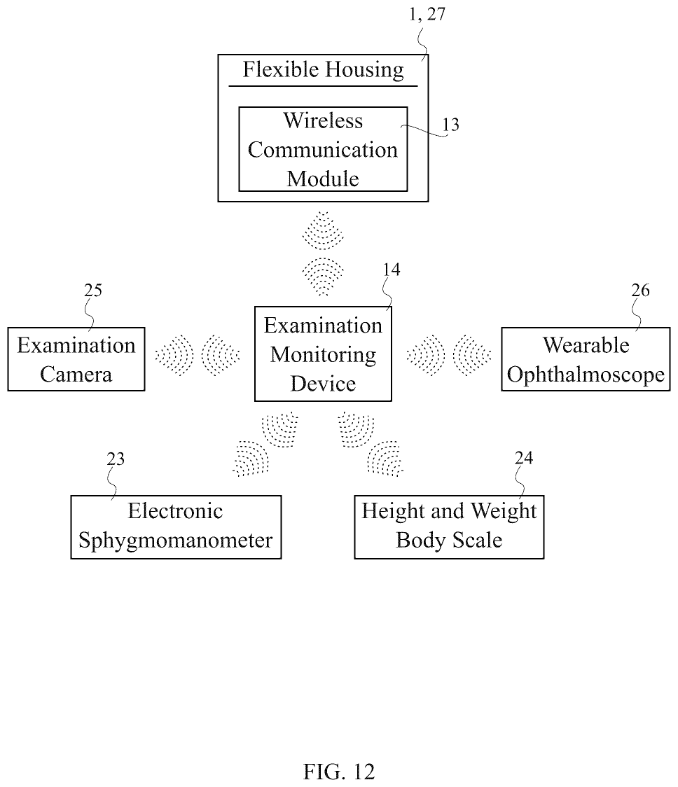

As previously discussed, the system of the present invention may include additional medical examination instruments that enable the remote physical examination of the patient along with the patient examination device 27. As can be seen in FIG. 12, in one embodiment, the system of the present invention may further comprise a height and weight body scale 24. The height and weight body scale 24 enables the automatic or semiautomatic measurement of the patient's height and weight. In general, the height and weight body scale 24 is positioned offset to the flexible housing 1 to not obstruct the operation of the patient examination device 27. In addition, the height and weight body scale 24 is communicably coupled with the examination monitoring device 14 to wirelessly relay the measured height and weight of the patient to the examination monitoring device 14. The height and weight body scale 24 can be implemented in different manners according to the environment where the remote examination process is being performed. For example, the height and weight body scale 24 can be provided as a standing scale that the patient can stand on during the remote examination process. The height and weight body scale 24 can also be integrated into the bed frame 22 to measure the height and weight of the bed-bound patient.

As can be seen in FIG. 12, in another embodiment, the system of the present invention may further comprise at least one examination camera 25 that enables the healthcare provider to remotely perform a visual examination of the patient during the examination process. The at least one examination camera 25 can be utilized to monitor different health vitals including, but not limited to, pallor, cyanosis, icterus, tremors, obvious masses, wounds respiratory rate, obvious bleeding, speech quality, and limb movements. In addition, the at least one examination camera 25 can include several camera sensors, such as an infrared sensor for fever and other abnormalities. In general, the at least one examination camera 25 is positioned offset to the flexible housing 1 to not obstruct the operation of the patient examination device 27. In addition, the at least one examination camera 25 is communicably coupled with the examination monitoring device 14 to wirelessly relay the video feed from the at least one examination camera 25 to the examination monitoring device 14.

As can be seen in FIG. 12, in another embodiment, the system of the present invention may further comprise a wearable ophthalmoscope 26. The wearable ophthalmoscope 26 is designed to be worn by the patient to enable the healthcare provider to perform remote eye examination of the patient. The wearable ophthalmoscope 26 can be a goggles-like device with a head band that secure the wearable ophthalmoscope 26 to the user's head during the remote examination process. In general, the wearable ophthalmoscope 26 is positioned offset to the flexible housing 1 to not obstruct the operation of the patient examination device 27. In addition, the wearable ophthalmoscope 26 is communicably coupled with the examination monitoring device 14 to wirelessly relay the captured eye data from the wearable ophthalmoscope 26 to the examination monitoring device 14. Further, like the patient examination device 27, the at least one examination camera 25, the electronic sphygmomanometer 23, the wearable ophthalmoscope 26, and the height and weight body scale 24 can include a corresponding serial number and ID. In other embodiments, additional medical examination instruments can be implemented, such as a volume evaluator for edemas or a robotics-assisted doppler component.

Although the invention has been explained in relation to its preferred embodiment, it is to be understood that many other possible modifications and variations can be made without departing from the spirit and scope of the invention.

What is claimed is:

1. A system for facilitating a remote medical examination, the system comprising:
a flexible housing;
a plurality of examination modules;
a controller;
a portable power source;
a wireless communication module;
an examination monitoring device;
the flexible housing comprising an outer housing surface, an inner housing surface, a first lateral edge, a second lateral edge, a proximal housing edge, and a distal housing edge;
the proximal housing edge and the distal housing edge being positioned opposite to each other across the flexible housing;
the first lateral edge and the second lateral edge being positioned opposite to each other across the flexible housing;
the first lateral edge and the second lateral edge being positioned in between the proximal housing edge and the distal housing edge;
the plurality of examination modules being distributed throughout and across the inner housing surface;
each of the plurality of examination modules being externally integrated onto the inner housing surface;
the controller, the portable power source, and the wireless communication module being mounted within the flexible housing;
the examination monitoring device being positioned offset to the flexible housing;
the plurality of examination modules and the wireless communication module being electronically connected to the controller;
the plurality of examination modules, the wireless communication module, and the controller being electrically connected to the portable power source; and
the wireless communication module being communicably coupled with the examination monitoring device.

2. The system as claimed in claim 1 further comprising:
each of the plurality of examination modules comprising a support base and a health vital monitoring device;
the support base being mounted onto the inner housing surface;
the health vital monitoring device being centered on the support base; and
the health vital monitoring device being mounted onto the support base, opposite the inner housing surface.

3. The system as claimed in claim 2, wherein the support base is a dome-shaped support base.

4. The system as claimed in claim 2, wherein the health vital monitoring device is a micro stethoscope.

5. The system as claimed in claim 2 further comprising:
a portable air pump;
wherein the support base is a pneumatic base;
the portable air pump being integrated into the flexible housing;
the portable air pump being in fluid communication with the support base of each of the plurality of examination modules;
the portable air pump being electronically connected to the controller; and
the portable air pump being electrically connected to the portable power source.

6. The system as claimed in claim 1 further comprising:
the plurality of examination modules being arranged into a rectangular array;
the rectangular array comprising a plurality of modules rows and a plurality of modules columns;
the plurality of modules rows being distributed from the proximal housing edge to the distal housing edge; and
the plurality of modules columns being distributed from the first lateral edge to the second lateral edge.

7. The system as claimed in claim 6, wherein the plurality of modules rows includes four rows of modules, and wherein the plurality of modules columns includes four columns of modules.

8. The system as claimed in claim 6 further comprising:
a housing fastener;
the housing fastener comprising a plurality of first fastener pieces and a plurality of second fastener pieces;
the plurality of first fastener pieces being distributed along the first lateral edge;
the plurality of first fastener pieces being mounted onto the outer housing surface, adjacent to the first lateral edge;
the plurality of second fastener pieces being distributed along the second lateral edge; and
the plurality of second fastener pieces being mounted onto the inner housing surface, adjacent to the second lateral edge.

9. The system as claimed in claim 8, wherein the housing fastener is a hook-and-loop fastener.

10. The system as claimed in claim 1 further comprising:
a bed frame;
the plurality of examination modules being arranged into a rectangular array;
the flexible housing being shaped into a C-shaped flexible housing;
the rectangular array comprising a plurality of modules rows and a plurality of modules columns;
the plurality of modules rows being distributed from the proximal housing edge to the distal housing edge;
the plurality of modules columns being distributed from the first lateral edge to the second lateral edge;
the rectangular array being positioned offset to the first lateral edge; and
the first lateral edge being laterally connected to the bed frame.

11. The system as claimed in claim 10, wherein the plurality of modules rows includes three rows of modules, and wherein the plurality of modules columns includes four columns of modules.

12. The system as claimed in claim 10 further comprising:
an electronic sphygmomanometer;
the electronic sphygmomanometer being positioned offset to the flexible housing;
the electronic sphygmomanometer being laterally mounted onto the bed frame; and
the electronic sphygmomanometer being communicably coupled with the examination monitoring device.

13. The system as claimed in claim 1 further comprising:
a height and weight body scale;
the height and weight body scale being positioned offset to the flexible housing; and
the height and weight body scale being communicably coupled with the examination monitoring device.

14. The system as claimed in claim 1 further comprising:
at least one examination camera;
the at least one examination camera being positioned offset to the flexible housing; and
the at least one examination camera being communicably coupled with the examination monitoring device.

15. The system as claimed in claim 1 further comprising:
a wearable ophthalmoscope;
the wearable ophthalmoscope being positioned offset to the flexible housing; and
the wearable ophthalmoscope being communicably coupled with the examination monitoring device.

16. The system as claimed in claim 1 further comprising:
a provider examination device;
at least one healthcare remote server; and
the provider examination device and the examination monitoring device being communicably coupled with the at least one healthcare remote server.

17. The system as claimed in claim 16, wherein the provider examination device is a wearable wireless earpiece.

18. The system as claimed in claim 16, wherein the at least one healthcare remote server includes at least one healthcare database.
